(19) United States
(12) Patent Application Publication
(10) Pub. No.: US 2021/0210321 A1
(43) Pub. Date: Jul. 8, 2021

(54) UNDERWATER GAS MEASUREMENT APPARATUS FOR GASES DISSOLVED IN WATER

(71) Applicant: Geomar Helmholtz-Zentrum fuer Ozeanforschung Kiel, Kiel (DE)

(72) Inventors: Roberto Enrique Benavides Noriega, Kiel (DE); Axel Berger, Kiel (DE)

(21) Appl. No.: 17/055,633

(22) PCT Filed: May 14, 2019
(86) PCT No.: PCT/DE2019/100437

§ 371 Date: Nov. 16, 2020

(30) Foreign Application Priority Data
May 24, 2018 (DE) 10 2018 112 526.7

(51) Int. Cl. H01J 49/04; G01N 1/40; G01N 33/18

(52) U.S. Cl. CPC H01J 49/0427; G01N 33/1886; G01N 1/4005

(57) ABSTRACT

An underwater gas measuring device for gases dissolved in water. The device can be used at great water depths, in particular in the deep-sea water column or on the sea floor. The underwater gas measuring device is better suited to perform a very large number of measurements than the pre-known measuring devices. The underwater gas measuring device preferably has at least one pressure tank downstream of the gas outlet with a gas outlet into the environment, the gas outlet being opened as soon as the internal pressure of the pressure tank exceeds the ambient pressure.

The invention relates to an underwater gas measuring device for gases dissolved in water. The device can also be used at great water depths, in particular in the deep-sea water column or on the sea floor.

Generic devices are known as scientific measuring instruments in oceanography and marine geology and as control devices for monitoring subsea installations of the hydrocarbon industry, e.g. pipelines and drilling platforms. In their simplest form, they comprise sampling devices with a number of sample bottles (usually 24-48 Niskin bottles maximum), which are filled individually at different times and are usually returned to the surface pressure-sealed. The measurement of the contained gases is then performed by gas chromatography in a laboratory, i.e. ex situ. The laboratory evaluation is time-consuming and expensive.

In addition, there is a great interest in in-situ measurements with a high resolution in time and exact location data, for example to detect gas distributions and gas leaks from oceanic sources. Such data can be investigated with compact underwater measurement systems that are attached to a submersible unit (“mooring”) or an ocean-bed hydrophone or a submersible vehicle (manned or remote-controlled or autonomous). For example, the emission of methane from seismically mapped gas hydrate deposits was first profiled in 1997 using in situ methane sensors mounted on submersible boats.

The measuring principles of in situ systems can be quite different. Early sensors, for example, were based on the reversible deposition of methane on tin dioxide with a change in electrical resistance or on the characteristic absorption of infrared light in carbon dioxide. Today, thanks to improved light sources, detectors and computing capacity, more advanced techniques such as mass spectroscopy (MS), Integrated Cavity Output Spectroscopy (ICOS) or Surface-Enhanced Raman Spectroscopy (SERS) are also available in small spaces. Also, common laser absorption spectroscopy has been developed with tunable and/or “exotic” wavelengths and allows for simultaneous determination of several gas species in one gas volume with high accuracy.

Some typical requirements for underwater measuring devices for dissolved gases can be summarized as follows:
Response time T₉₀ at most a few seconds, decay time not longer if possible;
no cross sensitivities of the sensor to different, simultaneously present gases (e.g. a typical problem with different alkanes);
no or at least calibratable dependencies of pressure, temperature and humidity;
robust against external pressure of up to 60 MPa (6000 m water depth);
lowest possible energy consumption (if no cable connection to the ship);
a very large number of in situ measurements should be possible.

All of the above-mentioned measuring principles are applicable in the gas phase only, i.e. the dissolved gas must always be separated from the water phase in a first step. Since the gas measuring device must be completely enclosed by a pressure vessel, typically a cylinder made of titanium or stainless steel, to protect it from the ambient pressure, a gas inlet opening is provided in the pressure vessel and sealed watertight with a semi-permeable membrane permeable to gas. Behind the membrane, a support device for the membrane against hydrostatic pressure is also arranged, for example a perforated or porous metal plate with open pore space, which is firmly connected to the pressure vessel, e.g. screwed. The membrane can consist of a synthetic fabric, e.g. polyester fleece, and is usually hydrophobically coated with silicone. Thus, the pressure vessel is watertight up to high pressures, but at a predetermined position it is not sealed against gas entry.

The design options for the gas inlet into the pressure vessel are diverse, and special reference is made to publication US 2009/0084976 A1, which provides very comprehensive proposals in this respect. The core of the publication is a deep-sea mass spectrometer for the analysis of various natural gases.

The principle of gas separation by the membrane and its supporting device at the gas inlet of the pressure cylinder is based on Henry's law, according to which the partial pressures in the gas phase must correspond to the dissolved concentrations in the water phase. In strict terms, this applies when an equilibrium has been established and the membrane does not create different diffusion barriers for different gas species. The latter must be investigated and, if necessary, taken into account in subsequent measurement evaluation procedures.

The adjustment of the equilibrium can be facilitated by targeted flow towards the gas inlet opening at relatively low volume rates of a few tens of milliliters per second. This has the effect of reducing the response time of the gas measuring device. However, if the gas measuring device is installed on a submersible vehicle, the flow to the gas inlet through the traveling movement may already be sufficient to shorten the response time.

It is also important to point out that a gas measuring device for underwater use based on the ICOS principle was developed by Wankel et al, “Characterizing the Distribution of Methane Sources and Cycling in the Deep Sea via in Situ Stable Isotope Analysis”, Environmental Science & Technology 2013 47 (3), 1478-1486 (DOI: 10.1021/es303661w), which is based on the ICOS principle and which can be used to measure not only the concentration of methane but also the ratio of stable carbon isotopes δ¹³C_{CH4}, allowing for conclusions to be made about the methane sources.

The two aforementioned publications describe gas measuring devices which comprise a measuring chamber in the pressure vessel which is in gas-exchanging communication with the supported membrane at the gas inlet of the pressure vessel. The partial pressures which are generated behind the membrane—i.e. within the pressure vessel—by diffusion of gas specimens from the ambient water should normally also be present in the measuring chamber without significant time delay.

In the measuring chamber any detection equipment for physical and/or chemical parameters of gas can be provided, in one example it would be a mass spectrometer and in the other one a laser light source and a detector are located at the end of a light path through the measuring chamber which is extended by mirroring. Other useful detection devices are sensors, which can also be additionally provided, and which are used to measure pressure, temperature and humidity in the measuring chamber.

Usually, detection devices provide analog electrical voltages as measured values or output signals, which are digitized and electronically processed and typically also provided with a time stamp. Sometimes the digital signals already directly describe the desired physical and/or chemical parameters of the gas in the measuring chamber. In some cases, an electronic evaluation device must numerically post-process the signals, e.g. calculate or compare them to previously tabulated data, in order to form derivative signals that correspond to the parameters. For example, laser light absorption measurement values have to be converted into partial pressure or concentration values by post-processing. It is useful to generate at least one signal or one derivative signal of each detection device as a representative of a detected parameter from the evaluation device and to feed it to a non-volatile data storage. If the underwater gas detection device is installed on a lander system or on a diving robot, there is often a cable connection to the operator e.g. onboard a ship. The measured data can be forwarded directly to a logging computer on the user's system via digital data transmission. Alternatively, the measured data can be transmitted acoustically without a cable connection if the receiving user is within receiving range. Compact and low-cost non-volatile data storage devices or data loggers are also common built-in standard components of most underwater measuring devices, because often no direct or immediate data transmission to the user is possible.

It is important to mention that the gas measuring devices of the two mentioned publications each have a vacuum pump to evacuate the measuring chamber.

Siphoning a gas sample from the measuring chamber reduces both the decay time and the response time by slightly increasing diffusion through the membrane. Additionally, it prevents the remeasurement of residues of the last gas sample, which would lead to an incorrect correlation of successive measurements. This is especially important if the gas measuring device is being moved for profiling purposes and in this process may encounter very different chemical conditions even over short distances, e.g. in the proximity of plumes.

It is also remarkable that the authors of the publications do not specify exactly where the extracted gas sample is actually taken to. This is a little surprising, because one cannot reasonably assume that the vacuum pump or any other gas pump would be able to blow the gas sample out of the pressure cylinder against the water pressure in the deep sea. Moreover, such a blow-out would have to perform considerable mechanical work and thus put a great load on the internal energy supply of the gas measuring device. In any case, neither US 2009/0084976 A1 nor the paper by Wankel et al. contain any reference to the disposal of the measured gas sample into the environment.

This justifies the conclusion that the gas sample is pumped from the measuring circuit into a collecting chamber inside the pressure vessel. In extreme circumstances, the entire inner space of the pressure vessel, which is not occupied by components of the gas measuring device, can serve as a collecting chamber. The state of the art therefore suggests the following design of the suction device, which is connected downstream of the measuring chamber with gas outlet:
a. a vacuum pump;
b. a non-return valve downstream of the vacuum pump, arranged in the direction of flow;
c. a collecting chamber for measured gas downstream of the non-return valve;
d. a pump control device which is designed to cause the vacuum pump to extract gas from the measuring chamber and to feed it to the collecting chamber.

The formation of the vacuum or negative pressure in the measuring chamber can be detected by sensors. The vacuum pump is designed to empty the measuring chamber to a high degree within a few seconds, whereas the diffusion through the membrane is much slower. The pump can also work against a—limited—overpressure, and in any case the gas pressure in the collecting chamber will increase with the number of measurements, i.e. the increasing number of gas samples that are extracted. A non-return valve is therefore installed downstream of the pump in the direction of flow, which only allows gas to flow from the pump outlet into the collecting chamber, but closes automatically against the overpressure in the collecting chamber when the pump is switched off. US 2009/0084976 A1 states in paragraph 0004: “[. . . ] there is a need for a submersible system to perform long-term series sampling of dissolved gases in a water column in the ocean depths (e.g., at depths greater than 2500 m).” The question arises how many measurements should be considered sufficient for this. Numerous devices anchored to the seafloor are deployed during a measurement campaign with a ship and are only recovered months or even a year later. At interesting locations—such as natural gas sources like “black smokers”—one or two measurements a day do not meet the requirements. Instead, one has to take into account the order of magnitude of several thousand measurements during a mission. This would also be very desirable for the profiling of gas distributions with submersible vehicles, since here a gas measurement should be made every few seconds if possible.

No further measurements can be carried out at the latest when the number of extracted gas samples becomes so large that the gas pressure in the collecting chamber exceeds the capacity of the vacuum pump.

The invention challenges the task of proposing an underwater gas measuring device which is better suited to perform a very large number of measurements than the pre-known measuring devices.

The task is solved by an underwater gas measuring device for gases dissolved in water comprising
a. a pressure vessel with a gas inlet opening in the wall of the pressure vessel;
b. a semi-permeable, gas-permeable membrane sealing watertightly arranged at the gas inlet opening;
c. a support device for the membrane against hydrostatic pressure;
d. a measuring chamber in the pressure vessel in gas exchanging communication with the membrane and having a gas outlet;
e. a detection device for detecting at least one physical and/or chemical parameter of gas in the measuring chamber;
f. an electronic evaluation device designed to detect at least one signal from the detection device representing at least one detected parameter and designed for digitized forwarding and/or for non-volatile digital storage of at least one signal and/or at least one derivative signal;
g. a vacuum pump connected downstream of the gas outlet of the measuring chamber;
h. a non-return valve downstream of the vacuum pump arranged in the direction of flow;
i. a collecting chamber for measured gas downstream of the non-return valve;
j. a pump control device which is designed to cause the vacuum pump to suck gas from the measuring chamber and to supply it to the collecting chamber; characterized in that
k. the collection chamber has a variable volume and a gas outlet;
l. a pressure release valve is arranged at the gas outlet of the receiving chamber, which opens when a predetermined gas pressure in the receiving chamber is exceeded;
m. mechanical means for changing the volume of the receiving chamber are provided which permit the volume of the receiving chamber to be increased to a predetermined maximum volume when gas is supplied, while maintaining the predetermined gas pressure;
n. a drive for the mechanical means for changing the volume is provided and is designed to cause the mechanical means, when the predetermined maximum volume is reached, to reduce the volume of the receiving chamber while blowing out the gas content through the pressure relief valve, wherein
o. a gas pipe leads the gas from the outlet of the pressure relief valve to a gas outlet opening in the wall of the pressure vessel

The depending claims specify advantageous solutions.

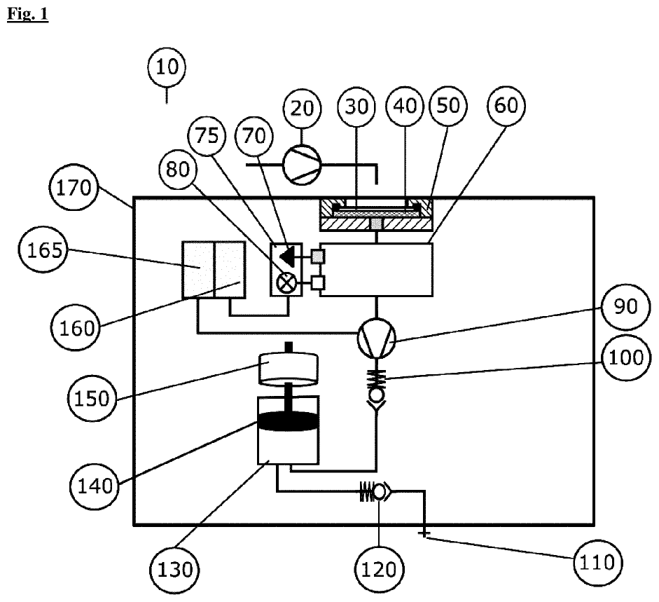

For further explanation of the invention, the single FIG. 1, which shows a sketch of the underwater gas measuring device with components designated therein, also serves as a detailed explanation of the invention. The components already known from the state of the art are in detail:
10 Ambient water
20 Inflow pump
30 Diaphragm
40 Support device for the diaphragm, e.g. metal disc
50 Diaphragm holder
60 Measuring chamber
75 Detection device (e.g. light source 70 and light detector 80)
90 Vacuum pump
100 Check valve
130 Collecting chamber
160/165 Evaluation device/Pump control device (processor)
170 Pressure vessel (with gas inlet opening on top)

The design of the underwater gas measuring device in accordance with the invention features a collecting chamber 130, which always maintains a constant gas pressure inside the device even during successive filling with extracted gas samples. The collecting chamber 130 is therefore expanded during filling with gas, i.e. its volume increases. When the collecting chamber 130 reaches a predetermined maximum volume, a driving mechanism 150 is activated, which compresses the collecting chamber 130 again by mechanical force. The contained gas is thereby blown out of the collecting chamber 130 through a gas pipe which leads to a gas outlet opening 110 in the wall of the pressure vessel 170.

Under deep-sea pressure conditions, blowing out should not take place into the surrounding water 10, but into a separate pressure tank (not shown), which is carried along specifically for the purpose of collecting the gas samples. The pressure tank is connected to the gas outlet of the gas measuring device via a pressure-resistant gas pipe, preferably a stainless steel pipe. The gas pipe is flange-mounted watertight at both ends, and in particular the gas outlet 110 of the gas measuring device is thus relieved of ambient pressure.

The pressure tank can be a comparatively simple, pressure-stable container, for example a closed hollow sphere or a closed hollow cylinder with hemispherical cover caps, and must only have a gas inlet and a gas outlet. Several pressure tanks can also be interconnected, so that, for example, the gas outlet of a first pressure tank is connected to the gas inlet of a second pressure tank by means of a pressure-resistant gas line. This gas line can be permanently open, so that the gas pressure between the different pressure tanks is always equalized. At least one pressure tank will have a gas outlet closed by a closed valve. Preferably, this valve allows the gas outlet to open automatically as soon as the ambient pressure drops below the gas pressure in the pressure tank. This is at least done when the gas measuring device is de-installed or recovered from great depths and is raised to sea level. It is considered safer to automatically discharge the unused gas which is pressurized in the pressure tank before the measuring device reaches a ship deck with people.

The underwater gas measuring device according to the invention therefore preferably has at least one pressure tank downstream of the gas outlet 110 with a gas outlet into the environment, the gas outlet being opened as soon as the internal pressure of the pressure tank exceeds the ambient pressure.

The connecting of pressure tanks to the gas measuring device is considered optional. If the gas measuring device is to be used only at water depths of a few tens of meters, for example in the Baltic Sea, the collecting chamber can be blown out into the surrounding water 10 without further action. In this case, the number of gas measurements which can be carried out would be limited only by the available energy, but no longer by the volume of the collecting chamber. In the deep sea, the number of possible measurements can be controlled by choosing the number and size of the pressure tanks, at least within certain limits. There is nothing to be said against evacuating at least one pressure tank before it is flanged to the gas measuring device and launched together with it.

It is considered an advantage of the invention that the same device is suitable for shallow water and deep sea measurements without modification of the internal measuring devices and installations. It is considered a further advantage that the vacuum pump 90 pumps against a constant predetermined gas pressure at any time when it sucks off a measured gas sample and feeds it to the collecting chamber 130. In particular, this allows the energy requirement of the pump 90 per measurement to be easily calculated.

The collecting chamber 130 is designed as a mechanical gas compressor.

A possible design, but not sketched in FIG. 1, may consist in a flexible balloon, e.g. made of a plastic foil, which is placed between two parallel, rigid plates, e.g. made of metal. When filled with gas, the balloon expands and pushes the plates apart. Once a maximum volume is reached, e.g. corresponding to a maximum distance between the plates, the actuator, preferably an electric motor, is activated, which compresses the plates again and thus reduces the volume of the balloon. In other words, the gas compressor can operate like a bellows.

As in FIG. 1, a piston compressor can be considered particularly preferable, the interior of which should form the collecting chamber 130. In a typical design, the piston compressor is a cylindrical tube, capped at one end, into the open end of which a piston 140 is inserted, which is airtight against the tube wall. If the capped side of the tube has at least one opening which serves as a gas inlet or gas outlet or both, the piston can suck gas into the chamber 130 or blow it out by mechanical movement—according to the same principle as a bicycle air pump.

It may be particularly advantageous for the purposes of the invention to use the interior 130 of a piston compressor as a collecting chamber when the piston compressor is arranged vertically, with the gas outlet located at the bottom of the compressor and the piston 140 pressing from above on the gas in the collecting chamber 130. The gas outlet can also serve as a gas inlet for gas coming from the vacuum pump 90. It is particularly preferred for the piston 140 to be freely movable in the compressor and to determine the pressure in the collecting chamber 130 under the effect of gravity. When the vacuum pump 90 feeds another measured gas sample into the collecting chamber 130, it works against the constant weight of the piston 140 and lifts it slightly. The gas pressure in the collecting chamber 130 is always the same and the pressure relief valve 120 at the gas outlet of the collecting chamber 130 remains closed. The piston 140 can be lifted to a predetermined height, which at the same time determines the maximum volume of the collecting chamber 130. Preferably, the piston compressor has a switching element (not shown) which is actuated when the interior 130 of the piston compressor reaches the predetermined maximum volume and which causes the actuator 150 to exert force on the piston 140 in the direction of the gas outlet. The actuator 150 can also be an electric motor which acts directly on the piston 140. For example, the switching element can be a mechanical element which is located above the piston head in the compressor, i.e. outside the collecting chamber 130. If the piston head is lifted above a predetermined height, it exerts a force on the switch, which is activated as a result. Activating the switch activates the drive 150 to exert a force on the piston 140. Preferably, the mechanical switching element jumps back to its initial position when the load is released, but this does not have to deactivate the drive 150.

Already a relatively small force leads to a compression of the gas volume located under the piston 140 in the collecting chamber 130 and increases the gas pressure in this chamber, which causes the pressure relief valve 120 to open and the collecting chamber 130 to be emptied by the gas discharge. If necessary, the gas can flow out into at least one pressure tank until piston 140 reaches a mechanical stop. Once the stop is reached, the actuator 150 can be deactivated and returned to its initial state. In particular, the gravity-controlled piston 140 is then freely movable again. The pressure relief valve 120 closes automatically after the overpressure has been reduced, and the collecting chamber 130 has reached its smallest volume. Further gas can be supplied by the vacuum pump 90 under the same conditions as before.

It is considered to be advantageous if the vacuum pump 90 is designed to create in the measuring chamber 60 a vacuum of not more than 100 hPa (=0,1 atmospheres=0,1 bar) with respect to an outlet gas pressure of at least 0.3 MPa (=3 atmospheres=3 bar) in a few seconds. It is also considered advantageous that the gas pressure in the collecting chamber 130 is at least 0.3 MPa. The higher the gas pressure is predetermined, the greater is the effective compression of the extracted gas sample and the more measurements can be carried out at the predetermined total volume of the pressure tanks.

If the receiving chamber is a cylindrical piston compressor with an internal diameter of 2 cm, then the freely movable piston 140 requires a mass of almost 10 kg in order to produce a gas pressure of 0.3 MPa in the receiving chamber 130 by its weight alone. However, an excessive total weight of the gas measuring device is not desirable for reasons of handling on board.

Alternatively, the drive 150 of the piston 140—or any other structural design of the gas compressor—can be controlled in such a way that it always maintains the predetermined gas pressure constant by active application of force in the receiving chamber 130. For this purpose, an additional pressure sensor in the collecting chamber 130 may be useful, whose measured values are fed to the control of the actuator 150—usually an electronic processor unit.

In an exemplary configuration, the underwater gas measuring device is installed on a platform which can be lowered to a desired measuring depth and provides a power supply for various measuring devices.

As already mentioned, the diffusion of the gas dissolved in ambient water through the membrane 30 at the gas inlet opening of the gas measuring device is accelerated by the fact that a flow at the membrane 30 is produced by a flow pump 20. The flow pump 20 can be designed as an integral part of the gas measuring device, which is then supplied with energy by the gas measuring device. Setting an equilibrium of the gas concentration in the measuring chamber 60 with respect to the ambient water 10 obeys Fick's diffusion law, i.e. the gas concentration follows an exponential function of time, and the final value can be calculated from the curve. It is therefore not absolutely necessary to wait for the t₉₀ measuring time to determine a measured value. The user can significantly reduce the actual measuring time if a higher measuring inaccuracy in return is acceptable.

### MOU1

In the example, the vacuum pump 90 has a delivery rate of more than 15 liters/min and generates a vacuum, a vacuum of less than 100 hPa, in a total gas volume of 25 milliliters, including the diaphragm holder 50 and measuring chamber 60 in less than 5 seconds. Due to the low gas volume, the time until the equilibrium in measuring cell 60 is set is reduced to t₉₀<15 s by evacuation.

After a measured value has been determined by the detection device 75—in the example with an optical measurement using a light source 70 and a light detector 80—and processed by the electronic evaluation device 160, the pump control device 165 activates the vacuum pump 90 to transfer the measured gas volume from the measuring chamber 60 to the collecting chamber 130. The collecting chamber 130 can be equipped with additional pressure sensors (not shown). As already described, the collecting chamber 130 is expanded in the process, whereby the pressure in the collecting chamber 130 must be kept constant.

In FIG. 1 the evaluation unit 160 and the pump control unit 165 are shown as one structural unit. This is not necessarily the case, however, it is advisable as the pump control unit 165 should only activate the vacuum pump 90 when the measurement of a gas volume has been completed and the measured data have been processed by the evaluation unit 160, i.e. recorded e.g. in the internal data logger or transferred to the user by cable. Thus, the two devices must communicate with each other and can usually be designed as conventional computer processors with software. It is unproblematic to implement devices 160 and 165 with a single processor.

In the embodiment example, the collecting chamber 130—as described above—is the interior of a piston compressor with a maximum volume of 20 milliliters. During the gas absorption the receiving chamber 130 is kept at a constant pressure of 0.4 MPa. When transferring the measured gas sample from the measuring chamber 60 to the collecting chamber 130, the vacuum pump 90 performs an initial compression, i.e. the gas sample is compressed by the pressure increase on its way. The volume of the collecting chamber 130 increases by significantly less than 25 milliliters, typically by about 5-6 milliliters, when a single gas sample is taken. The vacuum pump 90 in the embodiment example is designed to compress to an outlet pressure of up to 0.7 MPa.

The piston compressor in the embodiment example is—now as an alternative to the gravity-controlled piston compressor—equipped with a powerful drive 150, which can build up a gas pressure of up to 5 MPa in the interior 130 (second compression). The maximum pressure actually achieved in the compressor depends on the design of the pressure relief valve 120 at the gas outlet of the receiving chamber. The pressure relief valve 120 may be designed to open only at a gas pressure of 5 MPa.

The exemplary gas measuring device is therefore not subject to any limitation concerning the maximum number of measurements up to a measuring depth of approximately 500 m, since the measured gas can first be transferred to the collecting chamber 130 and then blown out into the ambient water 10 through the gas outlet 110. As already mentioned, the energy requirement for the piston compressor is not insignificant, so that a direct power supply from the research vessel is desirable.

For greater measuring depths, where the gas can no longer be blown out into the surrounding water 10, it is foreseen to connect at least one external tank. The exemplary external tank has an internal volume of 500 milliliters, which offers the possibility of holding at least one thousand (1000) gas samples, because the piston compressor can compress the original 25 milliliters of gas volume of a gas sample to a maximum of 0.5 milliliters in the external tank. The tank can withstand a maximum internal pressure of 5 MPa and an external pressure of 60 MPa.

The invention presented enables the construction of a gas measuring device which can be used to determine a large number of concentration data in ambient water very quickly and opens up the possibility of creating very accurate concentration profiles of different gases simultaneously.

1. An underwater gas measuring device for gases dissolved in water comprising:
a. a pressure vessel (170) with a gas inlet opening in the wall of the pressure vessel (170);
b. a semi-permeable, gas-permeable membrane (30) arranged at the gas inlet opening in a watertight manner;
c. a support device (40) for supporting the membrane (30) against hydrostatic pressure;
d. a measuring chamber (60) in the pressure vessel in gas exchanging communication with the membrane (30) and having a gas outlet;
e. a detection device (75) for detecting at least one physical and/or chemical parameter of gas in the measuring chamber (60);
f. an electronic evaluation device (160) designed to detect at least one signal of the detection device (75) representing at least one detected parameter and designed for digitized transmission and/or for non-volatile digital storage of at least one signal and/or at least one derivative signal;
g. a vacuum pump (90) connected downstream of the gas outlet of the measuring chamber (60);
h. a non-return valve (100) connected downstream of the vacuum pump (90) in the flow direction;
i. a collecting chamber (130) downstream of the non-return valve (100) for measured gas;
j. a pump control device (165) which is designed to cause the vacuum pump (90) to draw gas from the measuring chamber (30) and to supply it to the collecting chamber (130);
wherein
k. the collection chamber (130) has a variable volume and a gas outlet;
l. a pressure relief valve (120) is arranged at the gas outlet of the receiving chamber (130), which opens when a predetermined gas pressure in the receiving chamber
m. mechanical means (140) are provided for changing the volume of the receiving chamber (130) which, when gas is supplied, allow the volume of the receiving chamber (130) to be increased up to a predetermined maximum volume while maintaining the predetermined gas pressure;
n. a drive (150) is provided for the mechanical means (140) for changing the volume and designed to cause the mechanical means (140), when the predetermined maximum volume is reached, to reduce the volume of the collecting chamber (130) while blowing out the gas content through the pressure relief valve (120),
and
o. a gas pipe leads the gas from the outlet of the pressure relief valve (120) to a gas outlet opening (110) in the wall of the pressure vessel (170).

2. The device according to claim 1, further comprising at least one pressure tank downstream of the gas outlet opening (110) with a gas outlet to the environment, the gas outlet configured to open as soon as the internal pressure of the pressure tank exceeds the ambient pressure.

3. The device according to claim 1, wherein the collecting chamber (130) is designed as the interior of a piston compressor.

4. The device according to claim 3, wherein the piston compressor is arranged vertically, the gas outlet being arranged at the bottom of the compressor and the piston (140) pressing on the gas in the collecting chamber (130) from above.

5. The device according to claim 4, wherein the piston (140) is mounted freely movable in the compressor and determines the pressure in the collecting chamber (130) under the effect of gravity.

6. The device according to claim 3, wherein the piston compressor has a switching element which is actuated when the interior of the piston compressor assumes a predetermined maximum volume and which causes the drive (150) to exert force on the piston (140) in the direction of the gas outlet.

7. The device according to claim 3, wherein the predetermined gas pressure in the receiving chamber (130) is at least 3 bar (0.3 MPa).
